(19) United States
(12) Patent Application Publication
(10) Pub. No.: US 2022/0050116 A1
(43) Pub. Date: Feb. 17, 2022

(54) DIMERIC COLLAGEN HYBRIDIZING PEPTIDES AND METHODS OF USE THEREOF

(71) Applicant: University of Utah Research Foundation, Salt Lake City, UT (US)

(72) Inventors: Julian L. Kessler, Salt Lake City, UT (US); Michael S. Yu, Salt Lake City, UT (US)

(21) Appl. No.: 17/348,694

(22) Filed: Jun. 15, 2021

Related U.S. Application Data
(60) Provisional application No. 63/038,987, filed on Jun. 15, 2020.

(51) Int. Cl. G01N 33/68; C07K 1/14

(52) U.S. Cl. CPC G01N 33/6887; G01N 2333/78; C07K 1/14

(57) ABSTRACT

Disclosed are methods of enriching collagen fragments in a sample comprising combining a sample comprising collagen fragments with a composition comprising any one of the dimeric CHPs described herein, and wherein the first CHP and second CHP bind to and form a triple helix with a collagen fragment; and removing the bound collagen fragments from the dimeric CHP providing a product enriched with collagen fragments. Disclosed are methods of detecting collagen in a sample comprising enriching collagen fragments from a sample, wherein enriching the collagen fragments comprises combining a sample comprising collagen fragments with a composition comprising a dimeric CHP, wherein the dimeric CHP comprises a first CHP and a second CHP, one or more linkers, and a branch point, wherein the collagen fragments bind the dimeric CHP; and detecting the binding of the collagen fragments to the dimeric CHP.

### CROSS-REFERENCE TO RELATED APPLICATIONS

This application claims the benefit of U.S. Provisional Patent Application No. 63/038,987, filed on Jun. 15, 2020, which is incorporated by reference herein in its entirety.

### REFERENCE TO SEQUENCE LISTING

The Sequence Listing submitted Aug. 4, 2021 as a text file named “21101_0417U2_Updated_Sequence_Listing.txt,” created on Aug. 4, 2021, and having a size of 12,009 bytes is hereby incorporated by reference pursuant to 37 C.F.R. § 1.52(e)(5).

### BACKGROUND

Collagen fragments are useful biomarkers for monitoring the severity and progression of many diseases related to pathologic extracellular matrix (ECM) remodeling. As MMPs and Cathepsins degrade ECM, collagen fragments are released into the extracellular space and make their way into systemic circulation as potential biomarkers of collagen turnover. To date, several collagen fragments from blood and urine have been correlated with disease progression in liver and renal fibrosis, as well as in osteoarthritis, metastatic bone cancer, and osteoporosis. These biomarkers are almost universally derived from the terminal crosslinked regions of fibrous or network collagens, which are abundant in connective tissues and easy to detect from biological fluid. Other classes of collagens such as FACITs (e.g. types IX, XII, XIV) and MACITs (e.g. types XIII, XVII, XXIII) which are important to cellular function may produce more efficacious biomarkers, but are in extreme low abundance compared to structural collagens. Furthermore, although the triple helix is the hallmark structural feature of the collagen superfamily, fragments derived from the triple helical region have been largely ignored as biomarkers. This is because proteolytic degradation of the triple helix's repetitive GXY sequence produces numerous fragments with similar sequences that have low affinity to conventional antibodies.

Since collagen fragments are products of both normal and pathologic conditions, a panel of fragments rather than any individual fragment is more likely to indicate a specific pathology. This has prompted recent interest in the proteomic analyses of biological fluid, made possible largely by the advancement in liquid chromatography with tandem mass spectrometry (LC-MS/MS). Such analyses, however, cannot discriminate all compounds present in biological fluids (e.g. urine or serum) as the overwhelming level of off target signals can prevent detection of low abundance targets. Therefore, enrichment of the target compounds is essential for accurate LC-MS/MS analysis.

Collagen hybridizing peptides (CHPs) provide a unique opportunity to enrich collagen fragments from biological fluid for LC-MS/MS analysis. CHPs contain repeats of GPO amino acid motif which has the highest triple helical folding propensity among all natural amino acid sequences, allowing CHPs to bind tightly to denatured collagen strands through triple helical hybridization. Since binding occurs by folding into the native super-secondary protein structure rather than by conventional epitope recognition, CHPs have the potential to bind denatured fragments derived from the triple helical region of all collagen types and can do so with minimal sequence bias. Although CHPs are highly specific to collagen, there are major challenges for using them to efficiently capture collagen fragments. Unlike CHPs that are exclusively composed of GPO repeats, collagen a chains contain many non-GPO triplets which form unstable triple helices. To capture collagen fragments, conventional monomeric CHPs on solid support would have to bind two collagen fragments. However, such a process will produce an unstable triple helix containing two low-stability collagen chains, likely resulting in inefficient capture (FIG. 1). Additionally, at low fragment concentration such as that found in urine, binding would be slow since the encounter of three strands in forming triple helix would be rate limiting. Both limitations can be solved by using a dimeric form of CHP.

### BRIEF SUMMARY

Disclosed are methods of enriching collagen fragments in a sample comprising combining a sample comprising collagen fragments with a composition comprising any one of the dimeric CHPs described herein, and wherein the first CHP and second CHP bind to and form a triple helix with a collagen fragment; and removing the bound collagen fragments from the dimeric CHP providing a product enriched with collagen fragments.

Disclosed are methods of diagnosing a disease or injury involving collagen damage in a subject comprising detecting whether collagen is present in a sample obtained from the subject, wherein the detecting step comprises enriching collagen fragments from the sample, wherein the enriching step comprises combining the sample with a composition comprising any one of the dimeric CHPs described herein, wherein the first CHP and second CHP bind to and form a triple helix with a collagen fragment in the sample; detecting the binding of the (denatured) collagen fragments to the dimeric CHP; and diagnosing the subject as having a disease or injury involving collagen damage when collagen fragments bound to the dimeric CHP are detected.

Disclosed are methods of detecting collagen in a sample comprising enriching collagen fragments from a sample, wherein enriching the collagen fragments comprises combining a sample comprising collagen fragments with a composition comprising a dimeric CHP, wherein the dimeric CHP comprises a first CHP and a second CHP, one or more linkers, and a branch point, wherein the collagen fragments bind the dimeric CHP; and detecting the binding of the collagen fragments to the dimeric CHP.

Disclosed are methods of determining if a treatment is effective comprising detecting the amount of collagen in a sample obtained from the subject after treatment, wherein the detecting step comprises enriching collagen fragments from the sample, wherein the enriching step comprises combining the sample with a composition comprising one or more of the disclosed CHPs, wherein the dimeric CHP comprises a first CHP and a second CHP, wherein the first CHP and second CHP bind to and form a triple helix with a collagen fragment; detecting the binding of the collagen fragments to the dimeric CHP and quantifying the amount of collagen fragments bound to the dimeric CHP; and comparing the amount of collagen in a sample obtained from the subject after treatment with a control, wherein if the amount of collagen in a sample obtained from the subject after treatment is decreased compared to the control then the treatment is effective.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 is a schematic showing capturing collagen fragments by surface-immobilized collagen hybridizing peptides (CHPs).

FIGS. 2A, 2B, 2C, and 2D show triple helical folding and gelatin binding of M- and D-CHPs. (FIG. 1A) CD spectra at 4° C. showing the characteristic triple helix trace. (FIG. 1B) CD melting curves measured at 225 nm (heating rate: 0.5° C./min). (FIG. 1C) CD refolding at 4° C. monitored at 225 nm (M-CHP: 150 μM, D-CHP: 75 μM). (FIG. 1D) Fluorescently labeled M- and D-CHPs binding to crosslinked gelatin at 4° C. or 25° C.

FIGS. 3A and 3B show affinity of synthetic collagen fragments to surface-immobilized CHPs. (FIG. 3A) Amino acid sequences of synthetic collagen fragments, their locations in Rat Col1a1, and KD ¬against surface-immobilized D-CHP as calculated using curve fitting (4 parameter Hill slope). KD against M-CHPs were not calculated due to low binding. (FIG. 3B) Representative binding curves of synthetic collagen fragments binding to surface immobilized M- and D-CHPs. Additional binding curves are presented in FIG. 7.

FIGS. 4A and 4B show LC-MS/MS analysis of collagen fragments from mouse urine after enrichment by D-CHP functionalized beads. (FIG. 4A) Average number of unique collagen fragments (all samples combined) detected by LC-MS/MS mapped to each collagen type. Inset shows number of detected fragments from each mouse group (OVX or sham) with or without D-CHP enrichment. (FIG. 4B) Hierarchical clustering and heatmap of enriched collagen fragments in urine from OVX and sham-operated mice mapped to Col1a2, Col10a1, Col11a1, and Col13a1. Red color in dendrogram represents clustered OVX mice separated from sham-operated mice. Heatmap shows MS intensity of the detected collagen fragments (darker color indicates higher relative intensity) and their mapped locations along the four collagen types.

FIG. 5 shows SPR of gelatin binding to surface immobilized Biotin-M-CHP and Biotin-D-CHP, assessed at 37° C. Biotin-labeled CHPs were immobilized to neutravidin-displaying NLC sensor chips. Porcine gelatin (50 μg/mL) in running buffer (PBS with 0.1 mg/mL BSA and 0.01% TWEEN®20) was applied to the sensor surface during the association phase followed by elution with running buffer during the dissociation phase. Values are normalized to an unmodified lane blocked by biotin and to the RU intensity of each adsorbed CHP.

FIG. 6 shows CD melting curves of synthetic collagen fragments (150 μM, PBS) derived from CO1A1 RAT sequence. No melting transition was observed in any of the sequences.

FIG. 7 shows K_{D} curves for synthetic collagen peptides: Biotin-(GLT . . . GDK) (Top) and Biotin-(GEO . . . GEEGK) (Bottom), binding to surface-immobilized M- or D-CHPs.

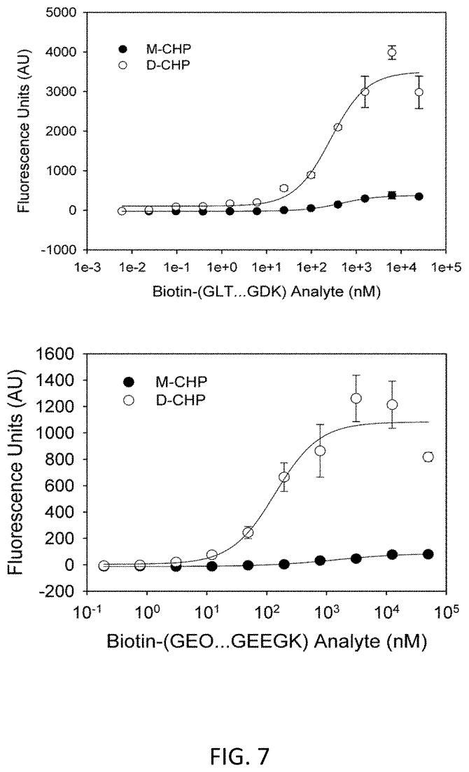

FIG. 8 shows ELISA-like binding assay of synthetic collagen fragment binding to surface bound D-CHP in urine. Samples were prepared by serial dilution of synthetic collagen fragment in urine and were applied to surface-immobilized D-CHPs, similar to the method described above. The curve represents the best fit curve from a 4 parameter Hill Slope with K_{D} at 110.5 nM.

FIGS. 9A and 9B show confirmation of OVX disease progression. (FIG. 9A) Uterine horn weight 4 weeks post-surgery, two-tailed, unpaired Welch's t tests. ***P=0.0002. (FIG. 9B) Bone mineral density (BMD) of OVX and sham-operated mice as determined by DXA in the metaphyseal region of the tibia. **P=0.0026.

FIG. 10 shows signals from collagen fragments. (Top) Number of peptide fragments detected by LC-MS/MS mapped to each collagen type with and without D-CHP enrichment. (Bottom) Fraction of MS intensity mapped to each type of collagen compared to total collagen intensity. Samples from OVX and sham-operated mice are combined in both graphs.

FIG. 11 shows fraction of M1 ion intensity of peptides mapped to collagen compared to all peptides detected by LC-MS/MS.

FIG. 12 shows clustering of all collagenous peptides detected by LC-MS/MS. Clustering is based on standardized ion intensity of all peptides detected that were mapped to a collagen sequence. Red indicates higher relative abundance, green indicates lower. Analysis of all collagen peptides detected was not able to clearly separate OVX from sham-operated mice. Therefore, individual collagen fragments were selected (FIG. 4B).

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a dimeric collagen hybridizing peptide” includes a plurality of such dimeric collagen hybridizing peptide, reference to “the dimeric collagen hybridizing peptide” is a reference to one or more dimeric collagen hybridizing peptides and equivalents thereof known to those skilled in the art, and so forth.

The term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” a disease or injury involving collagen damage can refer to reducing or eliminating the amount of damaged/denatured collagen. Treatment can also be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The term “subject” refers to any organism from which a sample is obtained and/or is the target of administration, e.g. an animal. Thus the subject of the disclosed methods can be a vertebrate, such as a mammal. For example, the subject can be a human. The term does not denote a particular age or sex. Subject can be used interchangeably with “individual” or “patient.”

As used herein, the terms “administering” and “administration” refer to any method of providing a one or more of the disclosed dimeric collagen hybridizing peptides, peptide conjugates, compositions or treatment (e.g. therapeutics) to a subject. Such methods are well known to those skilled in the art and include, but are not limited to: oral administration, transdermal administration, administration by inhalation, nasal administration, topical administration, intravaginal administration, ophthalmic administration, intraauralintramural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject.

As used herein, “prevent” or “prevention” is meant to mean minimize the chance that a subject who has an increased susceptibility for developing disease, disorder or condition will develop the disease, disorder or condition. For example, prevent as used herein can mean minimize the chance that a subject who has an increased susceptibility for developing a disease or injury involving collage damage will in fact get the disease or injury.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Dimeric Collagen Hybridizing Peptides

Disclosed are dimeric collagen hybridizing peptides (CHPs).

Disclosed are dimeric collagen hybridizing peptides comprising a first CHP and a second CHP, one or more linkers, and a branch point. In some aspects, the first CHP and second CHP comprise the sequence of at least (GXY)n (SEQ ID NO:1), wherein G is glycine, wherein X and Y are any amino acid, and wherein n is any number between 3 and 12. In some aspects, n can be any number between 2 and 50, between 3 and 30, or between 2 and 20.

In some instances, the first CHP and second CHP are identical. In some instances, the first CHP and second CHP are different. In some instances, the first CHP and second CHP can be different in the sense that the sequences are different or they can have the same sequence but the number of repeats (i.e. n) is different.

Disclosed are dimeric CHPs comprising a first CHP and second CHP; a linker; and a branch point, wherein the first CHP and second CHP comprise the sequence of at least (GXY)n (SEQ ID NO:1), wherein G is glycine, wherein X and Y are any amino acid, wherein n is any number between 3 and 12, and wherein X is proline, glutamic acid, or aspartic acid.

Disclosed are dimeric CHPs comprising a first CHP and second CHP; a linker; and a branch point, wherein the first CHP and second CHP comprise the sequence of at least (GXY)n (SEQ ID NO:1), wherein G is glycine, wherein X and Y are any amino acid, wherein n is any number between 3 and 12, wherein Y is a modified proline, lysine, or arginine. In some instances, X is proline, glutamic acid, or aspartic acid and Y is a modified proline, lysine, or arginine. A modified proline can be hydroxyproline or fluoroproline. In some aspects, X and Y can be any amino acid, wherein any amino acid comprises the standard twenty amino acids or a modified amino acids. In some aspects, a CHP with modified amino acids can be a peptoid. Thus, in some aspects, the first and/or second CHP is a peptoid. Peptoids, for example, are a class of peptidomimetics which comprise N-substituted glycine monomer units (Figliozzi et al, Synthesis of N-substituted glycine peptoid libraries. In Methods Enzymol., Academic Press: 1996; Vol. 267, pp 437-447; Bartlett et al., Proc. Natl. Acad. Sci U.S.A. 1992, 89, 9367-9371). Peptoids are an important class of sequence-specific peptidomimetics shown to generate diverse biological activities (Patch et al. In Pseudo-peptides in Drug Development; Nielson, P. E., Ed.; Wiley-VCH: Weinheim, Germany, 2004; pp 1-35; Miller et al. Drug Dev. Res. 1995, 35, 20-32; Murphy et al. Proc. Natl. Acad. Sci. U.S.A. 1998, 95, 1517-1522; Nguyen et al. Science 1998, 282, 2088-2092; Ng et al. Bioorg. Med. Chem. 1999, 7, 1781-1785; Patch et al. J. Am. Chem. Soc. 2003, 125, 12092-12093; Wender et al. Proc. Natl. Acad. Sci. U.S.A. 2000, 97, 13003-13008; Wu et al. Chem. Biol. 2003, 10, 1057-1063; Chongsiriwatana et al. Proc. Natl. Acad. Sci. U.S.S. 2008, 105, 2794-2799). Oligopeptoids can be designed to display chemical moieties analogous to the bioactive peptide side chains while their abiotic backbones provide protection from proteolytic degradation.

Disclosed are dimeric CHPs comprising a first CHP and second CHP; a linker; and a branch point, wherein the first CHP and second CHP comprise the sequence of at least (GXY)n (SEQ ID NO:1), wherein G is glycine, wherein X and Y are any amino acid, wherein n is any number between 3 and 12, wherein n can be 6 or 9. Disclosed are dimeric CHPs comprising a first CHP and second CHP; a linker; and a branch point, wherein the first CHP and second CHP comprise the sequence of at least (GXY)n (SEQ ID NO:1), wherein G is glycine, wherein X and Y are any amino acid, wherein n is any number between 3 and 12, wherein the dimeric collagen hybridizing peptide can be represented by the formula [(Gly-Pro-Hyp)₆-Gly-Gly-Gly]₂-Lys, (Gly-Pro-Hyp)₆-Gly-Gly-Gly-Lys-Gly-Gly-Gly-(Hyp-Pro-Gly)₆, or

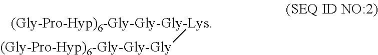

In some instances, the dimeric collagen hybridizing peptide comprises the formula [(Gly-Pro-Hyp)₉-Gly-Gly-Gly]₂-Lys, (Gly-Pro-Hyp)₉-Gly-Gly-Gly-Lys-Gly-Gly-Gly-(Hyp-Pro-Gly)₉, or

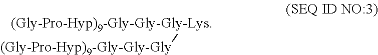

Disclosed are dimeric CHPs comprising a first CHP and second CHP; a linker; and a branch point, wherein the first CHP and second CHP comprise the sequence of at least (GXY)n (SEQ ID NO:1), wherein G is glycine, wherein X and Y are any amino acid, wherein n is any number between 3 and 12, wherein a glycine can be modified as an Aza-glycine. In some instances, only one glycine is modified as an Aza-glycine. In some instances, at least two glycines are modified as Aza-glycines. In some aspects, the X or Y can be Aza-glycines.

Disclosed are dimeric CHPs comprising a first CHP and second CHP; a linker; and a branch point, wherein at least one of the first CHP and second CHP comprises the sequence (Xaa₁-Xaa₂-Xaa₃)n¹-Xaa₄-Xaa₅-Xaa₆-(Xaa₇-Xaa₈-Xaa₉)n²(SEQ ID NOs:4), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine. In some instances, no more than one of Xaa₁, Xaa₂, Xaa₃, Xaa₄, Xaa₅, Xaa₆, Xaa₇, Xaa₈, or Xaa₉ can be aza-glycine. In some instances, Xaa₁, Xaa₂, and Xaa₃ are not the same amino acid. In some instances, Xaa₄, Xaa₅, and Xaa₆ are not the same amino acid. In some instances, Xaa₇, Xaa₈, and Xaa₉ are not the same amino acid. In some instances, at least two of Xaa₁, Xaa₂, and Xaa₃ are not the same amino acid. In some instances, at least two of Xaa₄, Xaa₅, and Xaa₆ are not the same amino acid. In some instances, at least two of Xaa₇, Xaa₈, and Xaa₉ are not the same amino acid.

Disclosed are dimeric CHPs comprising a first CHP and second CHP; a linker; and a branch point, wherein at least one of the first CHP and second CHP comprises the sequence (Xaa₁-Xaa₂-Xaa₃)n¹-Xaa₄-Xaa₅-Xaa₆-(Xaa₇-Xaa₈-Xaa₉)n²(SEQ ID NOs:4), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, wherein at least one of the first CHP and second CHP comprise the sequence (Gly-Pro-Hyp)₃-azGly-Pro-Hyp-(Gly-Pro-Hyp)₃ (SEQ ID NO:5), (Pro-Hyp-Gly)₃-Pro-Hyp-azGly-(Pro-Hyp-Gly)₃ (SEQ ID NO:6), or (Pro-Hyp-Gly)₃-Pro-Pro-azGly-(Pro-Hyp-Gly)₃ (SEQ ID NO:7).

Disclosed are dimeric CHPs comprising a first CHP and second CHP; a linker; and a branch point, wherein at least one of the first CHP and second CHP comprises the sequence (Xaa₁-Xaa₂-Xaa₃)n¹-Xaa₄-Xaa₅-Xaa₆-(Xaa₇-Xaa₈-Xaa₉)n²(SEQ ID NOs:4), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine, and at least one of Xaa₁, Xaa₂, Xaa₃, Xaa₄, Xaa₅, Xaa₆, Xaa₇, Xaa₈, or Xaa₉ is aza-glycine, wherein n¹ can be an integer from 1 to 20. In some instances, n² can be an integer from 1 to 20.

Disclosed are any of the disclosed dimeric CHPS, wherein the linker is between the collagen hybridizing peptides and the branch point. In some instances, there are at least two linkers. In some instances, the linker and branch point are on the C-terminal end of the first CHP and second CHP. In some instances, the linker and branch point are on the N-terminal end of the first CHP and second CHP. In some instances, the linker can be, but is not limited to, amino acid based or chemical. For example, the linker can be one or more glycine residues, aminohexanoic acid, or polyethylene glycol (PEG). The linker can vary depending on whether the peptides are linked at the N-terminal end or the C-terminal end. For example, for N-terminal linking a two cysteine linker can be used and for C-terminal linking a reactive end linker to a template molecule such as diacid can be used. Thus, disclosed are dimeric CHPs comprising a first CHP and second CHP; a linker; and a branch point, wherein the first CHP and second CHP comprise the sequence of at least (GXY)n (SEQ ID NO:1), wherein G is glycine, wherein X and Y are any amino acid, wherein n is any number between 3 and 12, wherein the linker is between the collagen hybridizing peptides and the branch point.

Disclosed are any of the disclosed dimeric CHPS, wherein the branch point is a molecule that links the first CHP and second CHP together through linkers attached to each first CHP and second CHP. The branch point can be amino acid based or a chemical compound. For example, in some instances, the branch point can be a lysine residue. In some instances, the branch point can attach to a linker which is attached to the first CHP and to a linker which is attached to the second CHP. Because the branch point attaches to a linker which attaches to the first CHP and second CHP, the branch point is present on whichever end of the peptides the linker is located on. Thus, the branch point can be either on the N-terminal end or C-terminal end of the CHPs. For example, disclosed are dimeric CHPs comprising a first CHP and second CHP; a linker; and a branch point, wherein the first CHP and second CHP comprise the sequence of at least (GXY)n (SEQ ID NO:1), wherein G is glycine, wherein X and Y are any amino acid, wherein n is any number between 3 and 12, wherein the branch point is a molecule that links the first CHP and second CHP together through linkers attached to each first CHP and second CHP.

In some aspects, the dimeric CHP is cyclic. For example, a linker and a branch point can be present at both the N-terminal end and the C-terminal end. Thus, in some aspects, the dimeric CHP can comprise at least two linkers and at least two branch points.

Disclosed are any of the disclosed dimeric CHPS, wherein the dimeric CHP can be attached or conjugated to a solid support. In some instances, the solid support can be attached via an attachment point present between the branch point and the solid support. In some instances, the attachment point can be any amino acid residue. In some instances, the branch point also serves as the attachment point for the solid support. For example, the attachment point can be a glycine residue. In some instances, solid supports can be, but are not limited to, resin, polymeric beads, agarose beads, nanotubes, nanoparticles, surface coated with gold, acrylamide, cellulose, nitrocellulose, glass, gold, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids or any polymeric surface. Solid supports can have any useful form including thin films or membranes, beads, bottles, dishes, fibers, optical fibers, woven fibers, chips, compact disks, shaped polymers, metals, particles and microparticles. A chip is a rectangular or square small piece of material.

Thus, disclosed are dimeric CHPs comprising a first CHP and second CHP; a linker; and a branch point, wherein the first CHP and second CHP comprise the sequence of at least (GXY)n (SEQ ID NO:1), wherein G is glycine, wherein X and Y are any amino acid, wherein n is any number between 3 and 12, wherein the dimeric CHP can be attached or conjugated to a solid support.

In some aspects, the dimeric CHPs do not bind native collagen.

In some aspects, the disclosed dimeric CHPs can be conjugated to an active agent forming a peptide conjugate. In some aspects, the disclosed peptide conjugates comprise an active agent, a spacer moiety, and a dimeric CHP. In some aspects, the dimeric CHP of the disclosed peptide conjugates can be any of the dimeric CHPs disclosed herein.

### C. Peptide Conjugates

Disclosed are peptide conjugates comprising an active agent, a spacer moiety, and a dimeric collagen hybridizing peptide, wherein the dimeric collagen hybridizing peptide comprises a first CHP and second CHP; a linker; and a branch point, wherein the dimeric CHP is one of the dimeric CHPs disclosed herein. In some aspects, the spacer moiety can be between the active agent and the first CHP or second CHP. In some instances, the spacer moiety can comprise aminohexanoic acid. In some instances, the spacer moiety can be one or more glycines or PEG. For example, disclosed are peptide conjugates comprising an active agent, a spacer moiety, and a dimeric collagen hybridizing peptide, wherein the dimeric CHPs comprise a first CHP and second CHP; a linker; and a branch point, wherein the first CHP and second CHP comprise the sequence of at least (GXY)n (SEQ ID NO:1), wherein G is glycine, wherein X and Y are any amino acid, wherein n is any number between 3 and 12.

In some aspects, the active agent can be a detectable moiety or a therapeutic agent. In some instances, the active agent can be attached to the N-terminal or C-terminal portion of at least one of the CHPs. In some instances, an active agent can be attached to only one of the CHPs. In some instances, an active agent can be attached to both of the CHPs. In some instances, an active agent can be present at both the N-terminal and C-terminal ends of one or both of the CHPs.

In some instances, the detectable moiety (or referred to as a detectable agent) can be a fluorescent dye, radioactive isotope, magnetic bead, metallic bead, colloidal particle, near-infrared dye, or an electron-dense reagent. Thus, detectable moieties can be, but are not limited to, fluorescent moieties, radioactive moieties, electronic moieties, and indirect moieties such as biotin or digoxigenin. When indirect moieties are used, a secondary binding agent that binds the indirect moiety can be used to detect the presence of a bound collagen hybridizing peptide. These secondary binding agents can comprise antibodies, haptens, or other binding partners (e.g., avidin) that bind to the indirect moieties.

In some instances, the therapeutic agent can be a therapeutic known to treat a disease or injury involving collagen damage. For example, the therapeutic agent can be, but is not limited to, any suitable pharmaceutical or other therapeutic agent, including but not limited to, osteogenic promoters, antimicrobials, anti-inflammatory agents, polypeptides such as recombinant proteins, cytokines or antibodies, small molecule chemicals or any combination thereof. In some instances, a therapeutic agent can be a cancer drug, arthritis drug or osteoporosis drug. Therapeutic agents can be capable of promoting bone growth, decreasing inflammation, promoting collagen stability. The therapeutic agent can include, but is not limited to, bone morphogenic protein (BMP), G-CSF, FGF, BMP-2, BMP-3, FGF-2, FGF-4, anti-sclerostin antibody, growth hormone, IGF-1, VEGF, TGF-.beta., KGF, FGF-10, TGF-.alpha., TGF-.beta.1, TGF-.beta. receptor, CT, GH, GM-CSF, EGF, PDGF, celiprolol, activins and connective tissue growth factors. In some instances, a therapeutic agent can be an antibody such as, but not limited to, Avastin, Eylea, Humira, ReoPro, Campath, tocilizumab, Ilaris, Removab, Cimzia, Erbitux, Zenapax, Prolia, Raptiva, Rexomun, Abegrin, HuZAF, Simponi, Igovomab, IMAB362, Imciromab, Remicade, Yervoy, Tysabri, Theracim, OvaRex, Vectibix, Theragyn, Omnitarg, Cyramza, Lucentis, Antova, Actemra, Herceptin, Ektomab, Stelara, HumaSPECT, HuMax-EGFr, HuMax-CD4. A therapeutic agent can target tumors, arthiritis, osteoporosis, MMP inhibitors, cathepsin inhibitors, interleukin inhibitors, TRAIL inhibitors, VEGF inhibitors, or CD binding agents.

In some instances, a disease or injury involving collagen damage can be, but is not limited to, cartilage/bone injury, tendon/ligament injury, corneal injury, and disease with high collagen remodeling activity such as cancer, arthritis, osteoporosis, fibrosis, kidney/bladder disease, and vulnerable plaques.

In some aspects, the disclosed peptide conjugates can be attached or conjugated to a solid support. In some instances, the solid support can be attached via an attachment point present between the branch point and the solid support. In some instances, the attachment point can be any amino acid residue. In some instances, the branch point also serves as the attachment point for the solid support. For example, the attachment point can be a glycine residue. In some instances, solid supports can be, but are not limited to, resin, polymeric beads, agarose beads, nanotubes, nanoparticles, surface coated with gold, acrylamide, cellulose, nitrocellulose, glass, gold, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids or any polymeric surface. Solid supports can have any useful form including thin films or membranes, beads, bottles, dishes, multiwell plates, fibers, optical fibers, woven fibers, chips, compact disks, shaped polymers, metals, particles and microparticles. A chip is a rectangular or square small piece of material.

### D. Compositions

Disclosed are compositions comprising one or more of the disclosed dimeric CHPs or peptide conjugates. In some instances, the disclosed compositions further comprise a pharmaceutically acceptable carrier. For example, disclosed are compositions comprising one or more dimeric CHPs, wherein the dimeric CHP comprises a first CHP and second CHP; a linker; and a branch point, wherein the first CHP and second CHP comprise the sequence of at least (GXY)n (SEQ ID NO:1), wherein G is glycine, wherein X and Y are any amino acid, and wherein n is any number between 3 and 12. Also disclosed are compositions comprising one or more dimeric CHPs, wherein the dimeric CHP comprises a first CHP and second CHP; a linker; and a branch point, wherein at least one of the first CHP and second CHP comprises the sequence (Xaa₁-Xaa₂-Xaa₃)n¹-Xaa₄-Xaa₅-Xaa₆-(Xaa₇-Xaa₈-Xaa₉)n²(SEQ ID NO:4), wherein Xaa₁, Xaa₂, Xaa₃, Xaa₄, Xaa₅, Xaa₆, Xaa₇, Xaa₈, Xaa₉ is glycine, proline, a modified proline or aza-glycine, and at least one of Xaa₁, Xaa₂, Xaa₃, Xaa₄, Xaa₅, Xaa₆, Xaa₇, Xaa₈, or Xaa₉ is aza-glycine.

1. Pharmaceutical Compositions

The compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, or conjugate of the invention is not compromised. Pharmaceutical compositions may also include one or more active ingredients (in addition to the composition of the invention) such as antimicrobial agents, anti-inflammatory agents, anesthetics, and the like.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the dimeric CHPs. Thus, compositions can be prepared for parenteral administration that includes dimeric CHPs dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The pharmaceutical compositions described above can be formulated to include a therapeutically effective amount of a composition disclosed herein. In some aspects, therapeutic administration encompasses prophylactic applications.

The pharmaceutical compositions described herein can be administered to the subject (e.g., a human subject or human patient) in an amount sufficient to delay, reduce, or preferably prevent the onset of clinical disease. Accordingly, in some aspects, the subject is a human subject. In therapeutic applications, compositions are administered to a subject (e.g., a human subject) already with or diagnosed with a disease or injury involving collagen damage in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effective amount includes amounts that provide a treatment in which the onset or progression of a disease or injury involving collagen damage is delayed, hindered, or prevented, or the autoimmune disease or a symptom of the autoimmune disease is ameliorated. One or more of the symptoms can be less severe. Recovery can be accelerated in an individual who has been treated.

The total effective amount of the conjugates in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

The pharmaceutical composition may be administered in a number of ways depending on whether local or systemic treatment is desired, and on the area to be treated.

### E. Methods of Enriching

Disclosed are methods of enriching collagen fragments in a sample comprising combining a sample comprising collagen fragments with a composition comprising any one of the dimeric CHPs described herein, and wherein the first CHP and second CHP bind to and form a triple helix with a collagen fragment; and removing the bound collagen fragments from the dimeric CHP providing a product enriched with collagen fragments. In some aspects, the collagen fragments are removed from the dimeric CHP by denaturing the triple helix. In some aspects the triple helix can be denatured by heat or other means including, but not limited to photo-destabilizing peptoid residues.

Disclosed are methods of enriching collagen fragments in a sample comprising combining a sample comprising collagen fragments with a composition comprising any one of the dimeric CHPs described herein, and wherein the first CHP and second CHP bind to and form a triple helix with a collagen fragment; and removing the bound collagen fragments from the triple helix providing a product enriched with collagen fragments. In some aspects, the collagen fragments are removed from the triple helix by denaturing the triple helix. In some aspects the triple helix can be denatured by heat or other means including, but not limited to photo-destabilizing peptoid residues.

Disclosed are methods of enriching collagen fragments in a sample comprising combining a sample comprising collagen fragments with a composition comprising any one of the dimeric CHPs described herein, and wherein the first CHP and second CHP bind to and form a triple helix with a collagen fragment; removing all unbound collagen fragments or other unbound components from the sample, removing the bound collagen fragments from the triple helix providing a product enriched with collagen fragments and optionally analyzing the collagen fragments. In some aspects, the collagen fragments are removed from the triple helix by denaturing the triple helix. In some aspects the triple helix can be denatured by heat or other means including, but not limited to photo-destabilizing peptoid residues.

Disclosed are methods of enriching collagen fragments in a sample comprising combining a sample comprising collagen fragments with a composition comprising any one of the dimeric CHPs described herein, and wherein the first CHP and second CHP bind to and form a triple helix with a collagen fragment; removing all unbound collagen fragments or other unbound components from the sample, and optionally analyzing the triple helix.

In some aspects, the collagen fragments comprise regions of intact triple helical collagen. For example, a portion of the collagen fragment can be denatured and a portion of the collagen fragment can be intact. In some aspects, the collagen fragments are denatured collagen fragments. Collagen fragments can be useful biomarkers for monitoring the severity and progression of many diseases related to pathologic extracellular matrix (ECM) remodeling. As MMPs and Cathepsins degrade ECM, collagen fragments are released into the extracellular space and make their way into systemic circulation as potential biomarkers of collagen turnover. In some aspects, the collagen fragments are derived from the triple helical region of one or more collagen types. In some aspects, the collagen fragments are from naturally occurring collagen. In some aspects, the collagen fragments are derived from native collagen but has denatured due to burns or mechanical or chemical denaturation. In some aspects, collagen fragments can come from any collagen type. Collagen hybridizing peptides (CHPs) provide an opportunity to enrich collagen fragments from a biological fluid for further analysis, including, but not limited to LC-MS/MS analysis. CHPs contain can contain repeats of GPO amino acid motif which has the highest triple helical folding propensity among all natural amino acid sequences, allowing CHPs to bind tightly to denatured collagen strands through triple helical hybridization. Since binding occurs by forming a triple helix between collagen fragments and CHPs rather than by conventional epitope recognition, CHPs can bind to denatured fragments derived from the triple helical region of all collagen types and can do so with minimal sequence bias.

In some aspects, using monomeric CHPs can cause two main issues for collagen fragment capture. In some instances, the collagen fragments captured can have low triple helical forming sequences. This can cause an unstable triple helix between the CHP and two collagen fragment peptides. In some aspects, for the CHP binding reaction to occur, two collagen fragments (found in low concentrations in serum) need to be present at the site of the bound monomeric CHP. Additionally, at low fragment concentration such as that found in urine, binding would be slow since the encounter of three strands in forming triple helix would be rate limiting. As disclosed herein, both limitations could be solved by using a dimeric form of CHP. As disclosed herein dimeric CHPs as an intermediate product during the synthesis of heterotrimeric collagen mimetic peptides, and these structures can hybridize to denatured collagen or collagen fragments. In some aspects, GPO triplets can form the most stable triple helices.

For example, disclosed are methods of enriching collagen fragments in a sample comprising combining a sample comprising collagen fragments with a composition comprising a dimeric CHP, wherein the dimeric CHP comprises a first CHP and a second CHP, one or more linkers, and a branch point, wherein the first CHP and second CHP comprise the sequence of at least (GXY)n (SEQ ID NO:1), wherein G is glycine, wherein X and Y are any amino acid, and wherein n is any number between 3 and 12, and wherein the first CHP and second CHP bind to and form a triple helix with a collagen fragment; and removing the bound collagen fragments from the dimeric CHP or triple helix thereby providing a product enriched with collagen fragments.

In some aspects, the collagen fragment that binds to the dimeric CHP to form a triple helix is a denatured collagen fragment. In some aspects, the collagen fragments comprise regions of intact triple helical collagen. For example, a portion of the collagen fragment can be denatured and a portion of the collagen fragment can be intact. In some aspects, the collagen fragments are denatured collagen fragments. In some aspects the collagen fragments are derived from the triple helical region of one or more collagen types. In some aspects the collagen fragments can be derived from any collagen type. In some aspects, the collagen fragments could be form any species that has collagen present.

In some aspects, the dimeric CHP is conjugated to a support. In some aspects, the dimeric CHP conjugated to a support can be any of those disclosed herein. For example, in some aspects, the support can be beads or a multiwell plate.

In some aspects, the dimeric CHP can be any of the dimeric CHPs disclosed herein. For example, in some aspects, the first CHP and second CHP are identical. In some aspects, the first CHP and second CHP are different.

In some aspects, the first CHP and second CHP comprise the sequence of at least (GXY)n (SEQ ID NO:1), wherein G is glycine, wherein X and Y are any amino acid, and wherein n is any number between 3 and 12. In some aspects, X is proline, modified proline, glutamic acid, or aspartic acid. In some aspects, Y is a modified proline, lysine, or arginine. In some aspects, one or more glycines is modified as an Aza-glycine.

In some aspects, the linker is between the collagen hybridizing peptides and the branch point. In some aspects, there are at least two linkers. In some aspects, the linker and branch point are on the C-terminal end of the first CHP and second CHP. In some aspects, the linker and branch point are on the N-terminal end of the first and second collagen hybridizing peptides. In some aspects, a linker and branch point are on both the C-terminal end and the N-terminal end of the first CHP and second CHP. For example, in some aspects, the dimeric CHP can be cyclic. In some aspects, the linker is one or more glycine residues, aminohexanoic acid, or polyethylene glycol (PEG). In some aspects, the branch point attaches to a linker which is attached to the first CHP and to a linker which is attached to second CHP. In some aspects, the branch point is a lysine residue.

In some aspects, the dimeric CHP comprises the formula

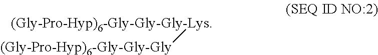

In some aspects, the dimeric peptide comprises the formula

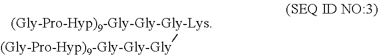

Disclosed are methods of enriching collagen fragments in a sample comprising combining a sample comprising collagen fragments with a composition comprising any one of the dimeric CHPs described herein, and wherein the first CHP and second CHP bind to and form a triple helix with a collagen fragment; removing the bound collagen fragments from the dimeric CHP providing a product enriched with collagen fragments; and further comprising determining the product enriched with collagen fragments. In some aspects, determining the composition (or make-up) of the product enriched with collagen fragments involves performing a peptidomic analysis on the product enriched with collagen fragments. In some aspects, determining the composition of the product enriched with collagen fragments involves performing a mass spectrometry on the product enriched with collagen fragments.

For example, disclosed are methods of enriching collagen fragments in a sample comprising combining a sample comprising collagen fragments with a composition comprising a dimeric CHP, wherein the dimeric CHP comprises a first CHP and a second CHP, one or more linkers, and a branch point, wherein the first CHP and second CHP comprise the sequence of at least (GXY)n (SEQ ID NO:1), wherein G is glycine, wherein X and Y are any amino acid, and wherein n is any number between 3 and 12, and wherein the first CHP and second CHP bind to and form a triple helix with a collagen fragment; removing the bound collagen fragments from the dimeric CHP providing a product enriched with collagen fragments; and further comprising determining the product enriched with collagen fragments. In some aspects, determining the composition of the product enriched with collagen fragments involves performing a peptidomic analysis on the product enriched with collagen fragments. In some aspects, determining the composition of the product enriched with collagen fragments involves performing mass spectrometry on the product enriched with collagen fragments.

In some aspects, the sample is a biological fluid. In some aspects, the biological fluid can be, but is not limited to, urine, blood, plasma, serum, saliva, interstitial fluid, mucus, or cerebrospinal fluid.

### F. Methods of Diagnosing

Disclosed are methods of diagnosing a disease or injury involving collagen damage in a subject comprising detecting whether collagen is present in a sample obtained from the subject, wherein the detecting step comprises enriching collagen fragments from the sample, wherein the enriching step comprises combining the sample with a composition comprising any one of the dimeric CHPs described herein, wherein the first CHP and second CHP bind to and form a triple helix with a collagen fragment in the sample; detecting the binding of the (denatured) collagen fragments to the dimeric CHP; and diagnosing the subject as having a disease or injury involving collagen damage when collagen fragments bound to the dimeric CHP are detected.

Disclosed are methods of diagnosing a disease or injury involving collagen damage in a subject comprising detecting whether collagen is present in a sample obtained from the subject, wherein the detecting step comprises enriching collagen fragments from the sample, wherein the enriching step comprises combining a sample comprising collagen fragments with a composition comprising a dimeric collagen hybridizing peptide (CHP), wherein the dimeric CHP comprises a first CHP and a second CHP, one or more linkers, and a branch point, wherein the first CHP and second CHP comprise the sequence of at least (GXY)n (SEQ ID NO:1), wherein G is glycine, wherein X and Y are any amino acid, and wherein n is any number between 3 and 12, and wherein the first CHP and second CHP bind to and form a triple helix with a collagen fragment; and removing the bound collagen fragments from the dimeric CHP providing a product enriched with collagen fragments; detecting the binding of the collagen fragments to the dimeric CHP; and diagnosing the subject as having a disease or injury involving collagen damage when collagen fragments bound to the dimeric CHP are detected.

In some aspects, the collagen fragment that binds to the dimeric CHP to form a triple helix is a denatured collagen fragment. In some aspects, the collagen fragments comprise regions of intact triple helical collagen. For example, a portion of the collagen fragment can be denatured and a portion of the collagen fragment can be intact. In some aspects, the collagen fragments are denatured collagen fragments. In some aspects the collagen fragments are derived from the triple helical region of one or more collagen types. In some aspects the collagen fragments can be derived from any collagen type. In some aspects, the collagen fragments could be form any species that has collagen present.

In some aspects, detecting the binding of the collagen fragments to the dimeric CHP can be performed while the collagen fragments are still bound to the dimeric CHP. In some aspects, detecting the binding of the collagen fragments to the dimeric CHP can be performed after removing the bound collagen fragments from the dimeric CHP or triple helix.

In some aspects, prior to the diagnosing step, a step of determining the composition (i.e. make-up) of the denatured collagen fragments. For example, in some aspects, determining the composition of the denatured collagen fragments can include performing peptidomic analysis on the enriched denatured collagen fragments. In some aspects, determining the composition of the denatured collagen fragments can include performing mass spectrometry. In some aspects, determining the composition of the denatured collagen fragments can be used as an indicator of a specific disease.

In some aspects, the disclosed methods of diagnosing further comprise administering an effective amount of a therapeutic to the diagnosed subject. For example, the presence of denatured collagen can result in diagnosing the subject as having osteoporosis. Thus, the therapeutic to be administered to the subject can be bisphosphonates. Any of the many known therapeutics for a disease or injury involving collagen damage can be administered.

In some aspects, the disclosed methods of diagnosing further comprise obtaining a sample from the subject prior to the step of detecting whether collagen is present in a sample.

In some instances, a disease or injury involving collagen damage can be, but is not limited to, cartilage/bone injury, tendon/ligament injury, corneal injury, and disease with high collagen remodeling activity such as cancer, arthritis, osteoporosis, fibrosis, and vulnerable plaques. Thus, any of the therapeutics known to treat these diseases can be administered after diagnoses.

In some aspects, the sample is a biological fluid. In some aspects, the biological fluid can be, but is not limited to, urine, blood, plasma, serum, saliva, interstitial fluid, mucus, or cerebrospinal fluid.

### G. Methods of Detecting

Disclosed are methods of detecting collagen in a sample comprising enriching collagen fragments from a sample, wherein enriching the collagen fragments comprises combining a sample comprising collagen fragments with a composition comprising a dimeric CHP, wherein the dimeric CHP comprises a first CHP and a second CHP, one or more linkers, and a branch point, wherein the collagen fragments bind the dimeric CHP; and detecting the binding of the collagen fragments to the dimeric CHP.

In some aspects, the collagen fragments comprise regions of intact triple helical collagen. For example, a portion of the collagen fragment can be denatured and a portion of the collagen fragment can be intact. In some aspects, the collagen fragments are denatured collagen fragments. In some aspects the collagen fragments are derived from the triple helical region of one or more collagen types.

In some aspects, detecting the binding of the collagen fragments to the dimeric CHP comprises removing any unbound compositions from the sample prior to detecting the binding of the collagen fragments to the dimeric CHP. In some aspects, removing unbound compositions from the sample can include washing the sample.

In some aspects, the dimeric CHP is conjugated to or attached to a solid support. For example, a solid support can be beads or a plate. When bound to a solid support, the dimeric CHPs can be washed to remove unbound collagen fragments. The detection of the collagen fragments can be performed using known direct or indirect detection methods. Direct detection can be, but is not limited to, amine detection or protein quantification. Indirect detection can be, but is not limited to, ELISA or ELISA-like assays.

In some aspects, the sample is a biological fluid. In some aspects, the biological fluid can be, but is not limited to, urine, blood, plasma, serum, saliva, interstitial fluid, mucus, or cerebrospinal fluid.

### H. Methods of Determining if a Treatment is Effective

Disclosed are methods of determining if a treatment is effective comprising detecting the amount of collagen in a sample obtained from the subject after treatment, wherein the detecting step comprises enriching collagen fragments from the sample, wherein the enriching step comprises combining the sample with a composition comprising one or more of the disclosed CHPs, wherein the dimeric CHP comprises a first CHP and a second CHP, wherein the first CHP and second CHP bind to and form a triple helix with a collagen fragment; detecting the binding of the collagen fragments to the dimeric CHP and quantifying the amount of collagen fragments bound to the dimeric CHP; and comparing the amount of collagen in a sample obtained from the subject after treatment with a control, wherein if the amount of collagen in a sample obtained from the subject after treatment is decreased compared to the control then the treatment is effective. In some aspects, the control is a sample from the subject prior to administering the treatment to the subject. For example, disclosed are methods of determining if a treatment is effective comprising detecting the amount of collagen in a sample obtained from a subject comprising administering a treatment to a subject, enriching collagen fragments from a sample from the subject after treatment, wherein the enriching step comprises combining the sample with a composition comprising one or more of the disclosed CHPs, wherein the dimeric CHP comprises a first CHP and a second CHP, wherein the first CHP and second CHP bind to and form a triple helix with a collagen fragment; detecting the binding of the collagen fragments to the dimeric CHP and quantifying the amount of collagen fragments bound to the dimeric CHP; and comparing the amount of collagen in the sample to a control sample obtained from the subject prior to administering the treatment, wherein if the amount of collagen in a sample obtained from the subject after treatment is decreased compared to the control then the treatment is effective

Disclosed are methods of determining if a treatment is effective comprising detecting the amount of collagen in a sample obtained from the subject after treatment, wherein the detecting step comprises enriching collagen fragments from the sample, wherein the enriching step comprises combining a sample comprising collagen fragments with a composition comprising a dimeric CHP, wherein the dimeric CHP comprises a first CHP and a second CHP, one or more linkers, and a branch point, wherein the first CHP and second CHP comprise the sequence of at least (GXY)n (SEQ ID NO:1), wherein G is glycine, wherein X and Y are any amino acid, and wherein n is any number between 3 and 12, and wherein the first CHP and second CHP bind to and form a triple helix with a collagen fragment; detecting the binding of the collagen fragments to the dimeric CHP and quantifying the amount of collagen fragments bound to the dimeric CHP; and comparing the amount of collagen in a sample obtained from the subject after treatment with a control, wherein if the amount of collagen in a sample obtained from the subject after treatment is decreased compared to the control then the treatment is effective. In some aspects, the control is a sample from the subject prior to administering the treatment to the subject.

In some aspects, the collagen fragments comprise regions of intact triple helical collagen. For example, a portion of the collagen fragment can be denatured and a portion of the collagen fragment can be intact. In some aspects, the collagen fragments are denatured collagen fragments. In some aspects the collagen fragments are derived from the triple helical region of one or more collagen types.

In some aspects, the control is the amount of collagen in a sample obtained from the subject prior to treatment. In some aspects, the control is the amount of denatured collagen in a sample obtained from the subject prior to treatment.

In some aspects, the sample is a biological fluid. In some aspects, the biological fluid can be, but is not limited to, urine, blood, plasma, serum, saliva, interstitial fluid, mucus, or cerebrospinal fluid.

### Examples

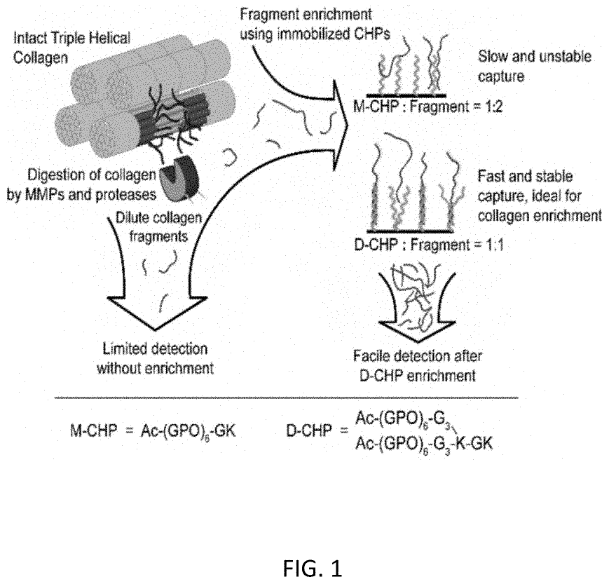

An end-tethered, dimeric CHP was produced to promote hybridization with dilute collagen fragments (FIG. 1). A dimeric CHP with sequence [Ac-(GPO)₆-G₃]₂-K-GK, designated as D-CHP, was designed to hybridize to collagen fragments via 1:1 stoichiometry. The peptide was synthesized by incorporating a parallel protected Fmoc-Lys(Fmoc)-OH residue during the Fmoc-mediated solid phase peptide synthesis (SPPS), and the two GPO chains were extended simultaneously after the branch point.

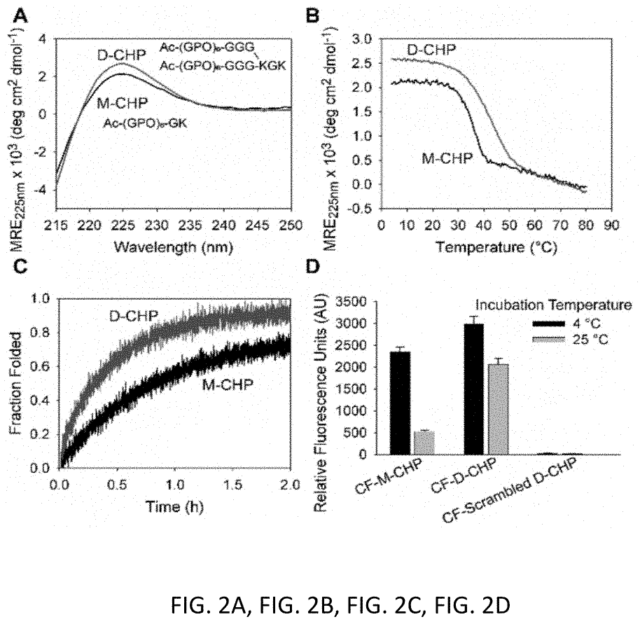

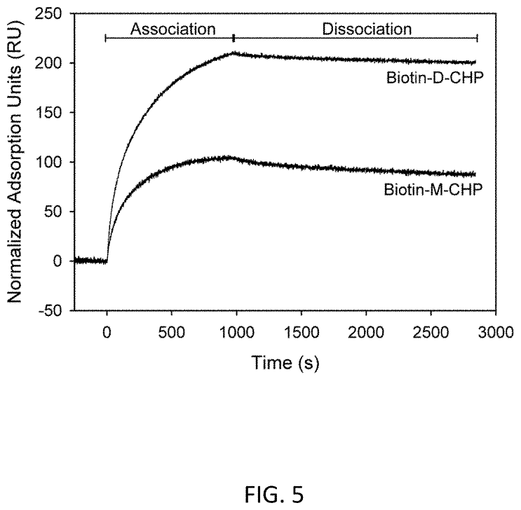

D-CHP's ability to fold into a triple helix was assessed using circular dichroism (CD) spectroscopy. As expected, D-CHP exhibited the signature triple helix CD trace and a clear first order melting transition at 44° C. which was 7° C. higher than that of the monomeric version of the CHP (M-CHP) (FIGS. 2A-B). At the same 150 μM strand concentration (as opposed to the CHP concentration), D-CHP showed faster folding (t_{1/2=18} min) than M-CHP (48 min) indicating that the two tethered strands of the D-CHPs cooperatively fold into a triple helix. To verify D-CHP's ability to hybridize with denatured collagen, melted, fluorescently labeled CHPs were applied to wells coated with crosslinked gelatin, followed by incubation at 4° C. or 25° C. At both conditions, D-CHP exhibited higher binding to the crosslinked gelatin than M-CHP, but the difference was larger at 25° C. In addition, comparative SPR experiments (immobilized CHP capturing dilute gelatin) demonstrated that D-CHP not only binds more gelatin but it does so with faster initial binding (FIG. 5). The results show that D-CHP produces a more stable complex with denatured collagen, presumably by folding into a hetero-triple helix comprised of two tethered CHP strands. D-CHP's fast refolding may not be suitable for targeting denatured collagens in tissues because such refolding abolishes collagen affinity; however as long as the D-CHPs are physically separated from each other and unable to fold inter-molecularly, fast folding would greatly enhance the capturing of dilute collagen fragments.

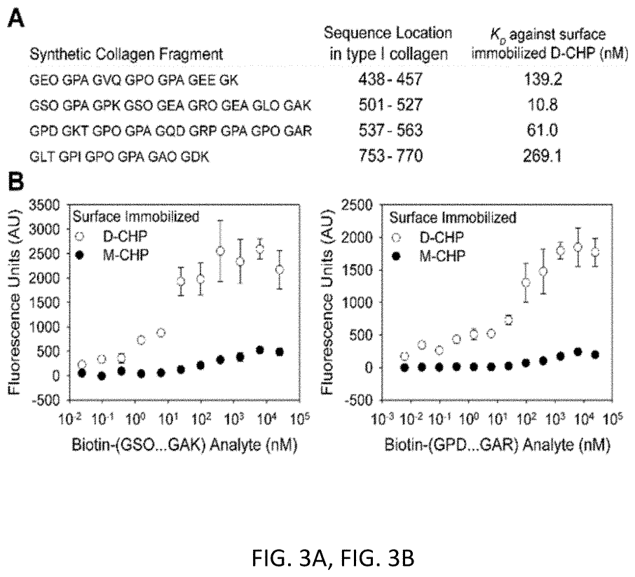

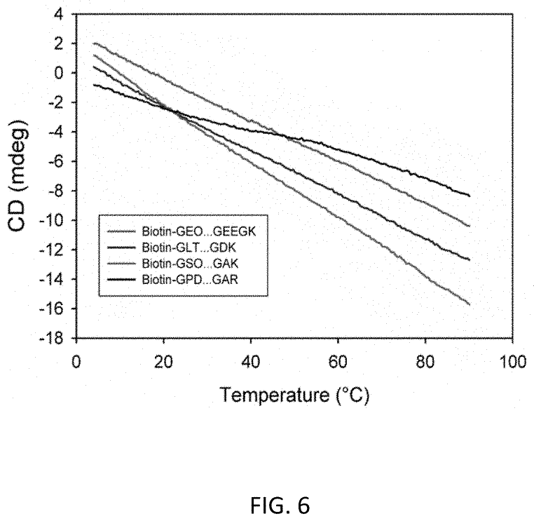

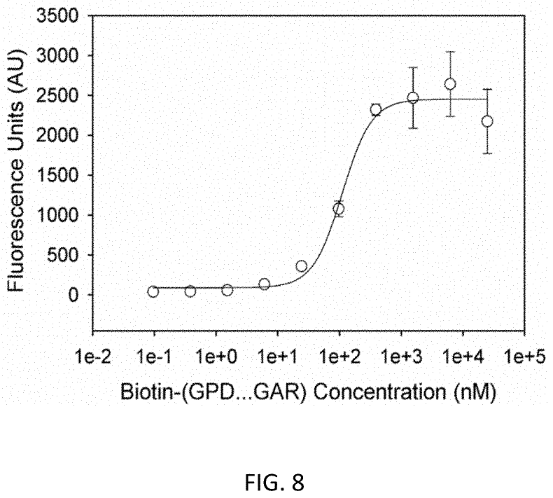

To investigate the affinity of collagen fragments to surface-immobilized M- and D-CHPs, an ELISA-like monolayer capture surface was prepared by covalently attaching CHPs to the surface of an amine reactive 96-well assay plate. Glycine was added during immobilization to spatially separate the CHPs and inhibit their intermolecular trimerization on the surface. To mimic collagen fragments, four peptides derived from the triple helical domain of the α-1 chain of rat type I collagen were synthesized. The synthetic collagen peptides were selected from domains lacking in consecutive GPO repeats, and covered a range of lengths and amino acid compositions (FIG. 3A). CD melting experiments confirmed that the synthetic collagen peptides were incapable of making homotrimers, as evidenced by no melting transition between 4° C. and 90° C. (FIG. 6). Despite having low triple helical propensity, all synthetic collagen peptides bound to D-CHP with KDs in the range of 10 to 270 nM, whereas their binding to M-CHP was negligible (FIG. 3B). The striking difference between D- and M-CHPs demonstrates the advantage of using D-CHP to capture collagen fragments. Since the synthetic collagen peptides have very low triple helical propensity, the two tethered GPO strands aid in their capture by increasing both the folding rate and the stability of the resulting triple helix. Surprisingly, D-CHP's capacity to capture synthetic collagen peptides was not affected even when the same experiments were conducted in urine (FIG. 8), demonstrating remarkably low non-specific binding of the CHPs which is consistent with previously reported works of staining protein gels and tissue sections.

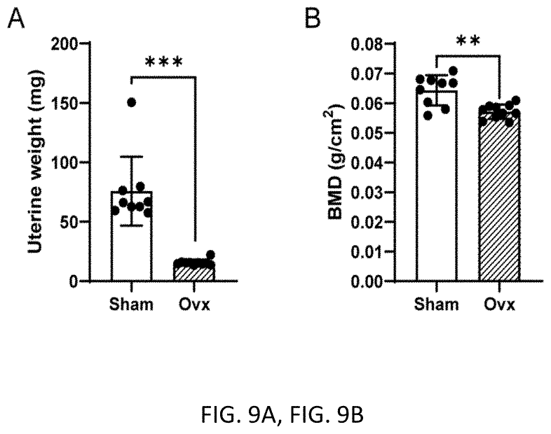

Encouraged by success in binding synthetic collagen peptides, solid supported D-CHP were used to enrich collagen fragments from urine to facilitate collagen peptidomic analysis. To produce beads capable of capturing collagen fragments from urine, D-CHP was prepared with a single biotin at the C terminus and it was immobilized to monomeric avidin beads. Urine was analyzed from a mouse model of post-menopausal osteoporosis, in which bilateral ovariectomy (OVX) leads to estrogen depletion, bone loss, and high collagen degradation activity (FIG. 9). To enrich collagen fragments, urine from OVX or sham-operated mice was mixed with D-CHP functionalized beads and incubated overnight at 4° C. The beads were then washed extensively to remove non-specifically adsorbed materials, followed by elution with 80° C. water which melts the triple helix and releases the bound collagen fragments. Unenriched urine samples were prepared using a conventional C-18 based extraction method which removes salts and non-protein components. Prepared urine samples were assessed using LC-MS/MS and the data were analyzed by an automated Mascot search against the SwissProt database (Taxonomy filter: Rodentia, no enzyme specificity) to yield peptide sequences. All detected peptides were screened against protein sequences from mouse collagen a chains to determine their collagen type of origin.

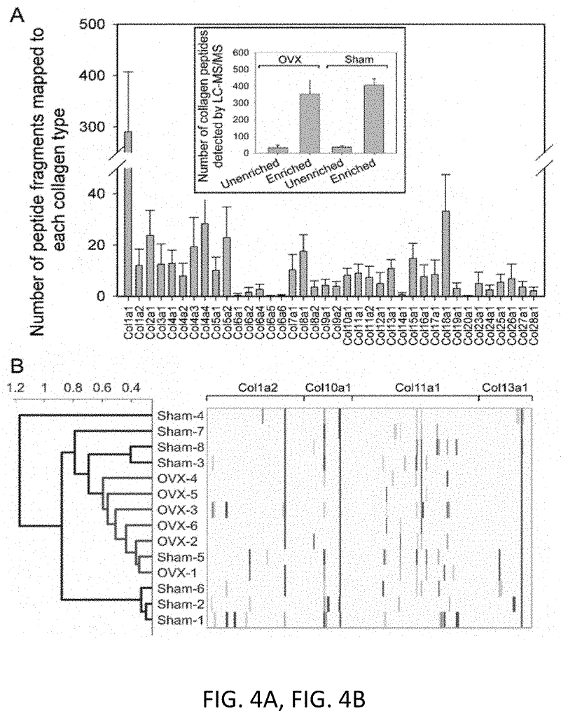

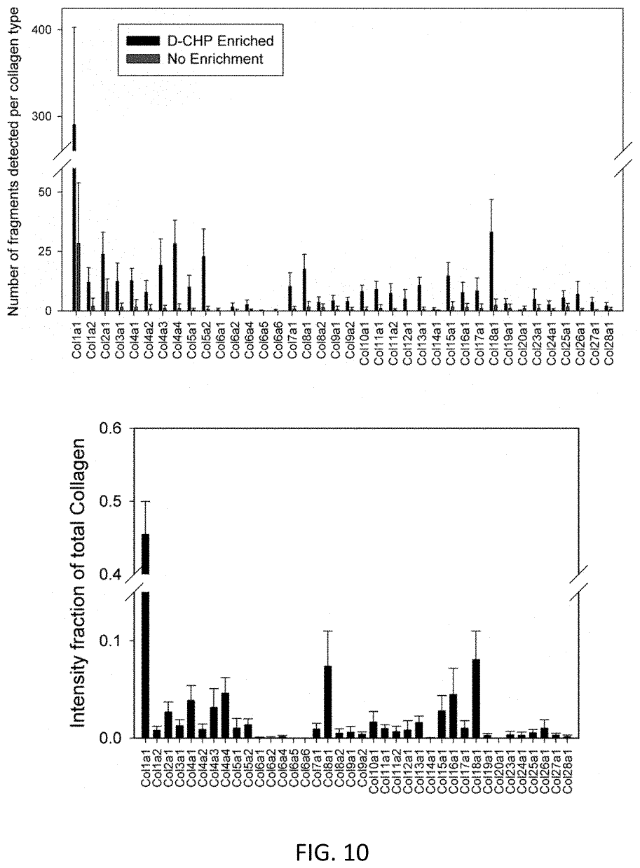

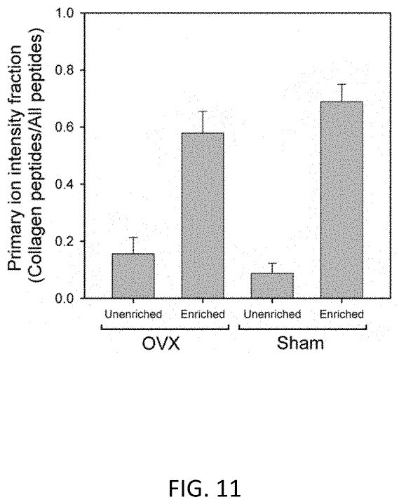

In the unenriched urine samples, collagen fragments represented only 12% of the total MS intensity with an average of 34 unique collagen fragments per sample. However, in samples enriched by D-CHP, close to 64% of the total MS intensity belonged to collagen and the number of collagen fragments detected increased to 383 per sample, which is an 11.2-fold increase (FIG. 4A, inset). In addition, peptide fragments were mapped onto all of the 38 collagen a strands, including FACITs and MACITs which are infrequently detected in biological fluid (FIG. 4A). Since the osteoporotic condition is associated with increased collagen degradation, the overall collagen signal was expected to be higher in the OVX samples. It was surprising to find that after D-CHP enrichment, both the total MS intensity and the number of detected collagen fragments were similar between OVX and sham-operated mice. This can be caused by saturation of D-CHPs on the beads during the enrichment process. In fact, in the unenriched samples, signals from collagen fragments were higher in OVX mice compared to sham-operated mice (FIGS. 10-11).

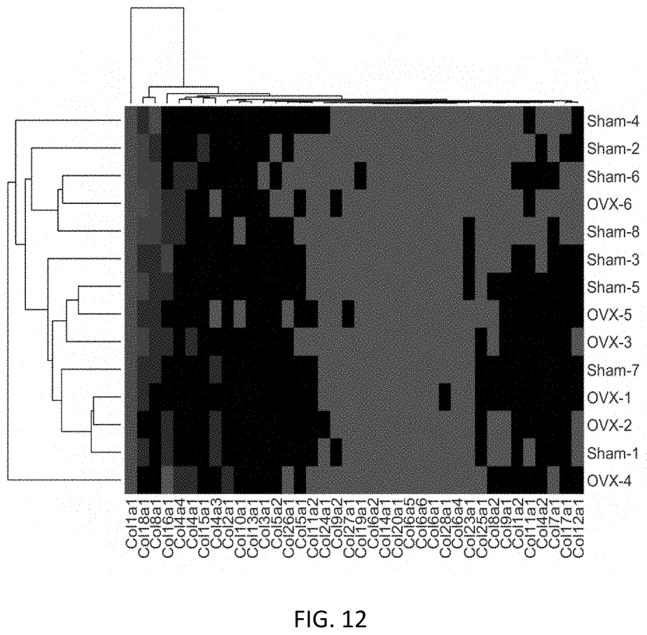

The difference between OVX and sham-operated mice was determined based on the intensities of individual collagen fragments. A clustering analysis of all collagen fragments detected from the enriched samples resulted in little separation between the OVX and sham-operated groups (FIG. 12). This is understandable since collagen degradation occurs under normal condition and many collagen fragments likely represent normal collagen remodeling rather than OVX pathology. However, when the peptide fragments from only Col1a2, Col10α1, Col11α1, and Col13α1 were analyzed, clustering of all OVX mice separated from all but one of the sham-operated mice, with more scattered clustering in the sham-operated mice was observed (FIG. 4B). Interestingly, each of these collagens is directly related to osteoporosis or bone remodeling. Col1 is the major organic component of the bone and is heavily degraded during bone resorption. Col13 is a MACIT collagen known to directly affect bone formation and is upregulated in osteoporosis. Col10 and Col11 are involved in endochondral ossification which is one of the bone healing responses known to be altered after OVX induced osteoporosis. Additional experiments using a large number of samples are required before this work can be used to predict pathology, but the results clearly demonstrate that collagen enrichment using D-CHPs can help identify a panel of useful collagen biomarkers which may otherwise go undetected. This work can be particularly suited for assessing disease near the kidney and bladder (e.g. renal fibrosis or bladder cancer) where urine is produced and stored. The same CHP-mediated enrichment strategy can be applied to tissue biopsies to improve collagen fragment detection.

1. Materials and Methods

i. Reagents

All reagents were used as received without further purification. Tentagel-R-RAM resin was purchased from Peptides International. Fmoc-Hyp(Trt)-OH was purchased from Novabiochem. NaCl, 10×PBS, NMP, DMF, TFA, diethyl ether, and QuantaBlu™ fluorogenic peroxidase kit were purchased from Thermo-Fisher Scientific. CF, piperidine, TWEEN®20, and BSA were purchased from Sigma Aldrich. DIEA was purchased from EMD Millipore. Dde-Lys(Fmoc)-OH, Fmoc-Lys(Fmoc)-OH, Fmoc-Ser(tBu)-OH, HATU, and HBTU were purchased from Chem-Impex International. d-Biotin was purchased from AnaSpec. SDS was purchased from JT Baker. Neutravidin-HRP was purchased from Life Technologies. SoftLink™ Soft Release Avidin Resin was purchased from Promega. All other solvents and reagents were purchased from AAPPTec LLC.

ii. Instrumentation

Automatic SPPS was performed on an AAPPTec Focus XC automatic peptide synthesizer. HPLC was performed using an Agilent SD-1 Prepstar HPLC Pump and a Zorbax 300SB-C18 column (Agilent). MALDI-TOF MS was performed using a Bruker MALDI-TOF UtrafleXtreme with CHCA used as the matrix for peptides with calculated masses less than 3 kDa, and SA as the matrix for peptides with calculated masses greater than 3 kDa. Peptides were lyophilized on a Labconco Freezone 4.5 freeze dry system. CD measurements were performed using a Jasco J-1500 Circular Dichroism Spectrometer with Julabo AWC100 temperature controller. Fluorescence from gelatin binding was measured on a SpectraMax M2e plate reader. SPR measurements were performed on a ProteOn™ XPR Protein Interaction Array System using ProteOn™ NLC Sensor Chips (BioRad). DXA scans were conducted using a Norland pDEXA densiometer (Norland Medical Systems). LC-MS/MS was performed by the Mass Spectrometry and Proteomics core facility at the University of Utah using an Eksper nanoLC 400 (Eksigent Technologies) with attached MAXIS II ETD Q-ToF mass spectrometer (Bruker).

iii. Synthesis and Purification of Peptides

All peptides were synthesized by Fmoc-mediated SPPS using an automated peptide synthesizer except for some intermediate and final coupling reactions which were run by manual SPPS, as noted below. Peptides were made on a Tentagel-R RAM resin (90 μm, 0.18 mmol/g). Resins were swelled in DMF for at least 30 min prior to the first reaction and at any step which involved the use of dry resin.

iv. Automated SPPS

a. Resin Preparation

Resin was added to the automatic SPPS vessel at an amount of 833 mg (0.15 mmol, 1 eq) for M-CHPs or 416 mg (0.075 mmol, 0.5 eq) for D-CHPs. The first Fmoc deprotection was performed by adding 10 mL of deprotection solution (20% piperidine in DMF) to the vessel followed by 5 min of mixing. The process was repeated with 10 min mixing. Following initial deprotection, the resin was washed with 10 mL of NMP 5 times.

b. Amino Acid Coupling

Stock solutions of Fmoc-protected amino acids (0.2 M in DMF), coupling solution (0.4 M HBTU, 0.4 M Cl-HOBt in DMF), and DIEA solution (2 M in NMP) were prepared and loaded to the automatic synthesizer. A single amino acid coupling proceeded as follows: Fmoc-protected amino acid stock solution (3.5 mL, 4.7 eq), coupling solution (1.7 mL, 4.5 eq), and DIEA solution (0.7 mL, 9.3 eq) were mixed and allowed to activate for 1 min. The mixture was then added to the resin and allowed to mix for 2 h. The reaction vessel was drained and the resins were washed with NMP 4× followed by a single wash with DMF. Fmoc protecting group was removed as described above and the resin was washed with NMP (4¬). Cycles were repeated until a full-length peptide was produced or a manual coupling step was required. Unless noted otherwise, following Fmoc-protected amino acids were used: A: Fmoc-Ala-OH, D: Fmoc-Asp(OtBu)-OH, E: Fmoc-Glu(OtBu)-OH, G: Fmoc-Gly-OH, I: Fmoc-Ile-OH, K: Fmoc-Lys(Boc)-OH, O: Fmoc-Hyp(tBu)-OH, P: Fmoc-Pro-OH, Q: Fmoc-Gln(Trt)-OH, R: Fmoc-Arg(Pbf)-OH, S: Fmoc-Ser(tBu)-OH, T: Fmoc-Thr(tBu)-OH, and V: Fmoc-Val-OH. For lysine residues which form the branch point in D-CHP sequences, Fmoc-Lys(Fmoc)-OH was used and subsequent couplings were performed to extend the two CHPs chains in parallel.

2. Manual SPPS

Automatic synthesis was paused after Fmoc deprotection of the previous amino acid and the resin was transferred to a manual synthesis vessel. The resin was washed with DMF (4¬). For all manual coupling steps described below, small scale reactions were performed when possible to conserve reagents, and calculations were made such that peptide on resin=1 eq. After manual coupling, the peptides were either cleaved from the resin or transferred to automated synthesizer for further coupling(s). For automated peptide syntheses continued after manual synthesis steps, the amount of reagents was not adjusted for the smaller quantity of resin. Therefore, the molar equivalence of the reagents was higher than what is described above in the automated SPPS section.

i. Biotin Coupling

d-Biotin (5 eq), HATU (5 eq), and HOAt (5 eq) were dissolved in NMP so that each component had a concentration of 0.16 M. The solution was added to the resin (1 eq peptide) followed by DIEA (7.5 eq) and was mixed for 2 h at room temperature. The reaction mixture was drained and resin was washed with DMF

ii. Ahx Coupling

Fmoc-Ahx-OH (5 eq), HATU (5 eq), and HOAt (5 eq) were dissolved in NMP so that each component had a concentration of 0.16 M. The solution was added to the resin (1 eq peptide) followed by DIEA (7.5 eq) and was mixed for 2 h at room temperature. The reaction mixture was drained and resin was washed with DMF (4¬). Piperidine in DMF (20% solution, 5 mL) was added to the resin and mixed for 30 min to remove the Fmoc protecting group. The resin was then washed with DMF

iii. CF Coupling

CF (6 eq) and PyAOP (6 eq) were dissolved in NMP so that each component had a concentration of 0.19 M. The solution was added to the resin (1 eq peptide) followed by DIEA (12 eq) and was mixed for 2 h at room temperature. Piperidine in DMF (20% solution, 5 mL) was added to the resin and mixed for 30 min to remove the Fmoc protecting group. The resin was then washed with DMF (4×).

iv. Lys(Biotin) and Lys(CF) Coupling

Dde-Lys(Fmoc)-OH (5 eq), HATU (5 eq), and HOAt (5 eq) were dissolved in NMP so that each component had a concentration of 0.16 M. The solution was added to the resin (1 eq peptide) followed by DIEA (7.5 eq) and was mixed for 2 h. The reaction mixture was drained and resin was washed with DMF (4×). Piperidine in DMF (20% solution, 5 mL) was added to the resin and mixed for 30 min to remove the Fmoc protecting group. The resin was washed with DMF (4×). Biotin or CF was coupled to the lysine's deprotected side chain using the same procedure as described above. Hydrazine in DMF (3%, 5 mL) was added and mixed for 15 min to cleave the Dde protecting group. The resin was then washed with DMF (4×).

v. Acetyl Capping

The capping procedure for all peptides was performed using manual SPPS. A capping solution of acetic anhydride (50 eq, 1 M) and DIEA (50 eq, 1 M) in DMF was added to the resin (1 eq peptide) and allowed to mix for 30 min. The resin was then washed with DMF (4×).

vi. Cleavage from Solid Support and Removal of Protection Groups

The Fmoc protecting group was removed as described above (if necessary) and the resin was washed with DMF (4¬) then DCM (4¬). The full length peptides were cleaved from solid support by addition of 8 mL cleavage cocktail containing TFA, H2O, and TIPS at a respective volume ratio of 95:2.5:2.5 followed by stirring for 2 h at room temperature. For small scale syntheses, 1 mL of the same cleavage cocktail was used. For peptides containing arginine, cleavage time was overnight (15 h).

3. Purification

Following SPPS, peptides were precipitated in cold diethyl ether. Precipitated peptides were isolated by centrifugation, decanting of the supernatants, followed by a second round of suspension in diethyl ether, centrifugation, and discarding of supernatant. Excess ether was evaporated and peptides were dissolved in H₂O and stored at 4° C. Crude peptides were then purified using reverse-phase HPLC equipped with a column heater (set at 70° C.), a mobile phase gradient of 5-35% acetonitrile in H₂O (0.1% TFA) with a flow rate of 4 mL/min. Peptide purity was verified using MALDI-TOF MS. Purified products were lyophilized and stored at 4° C.

4. Circular Dichroism

i. Peptide Solution Preparation

Stock peptide solutions were prepared by dissolving solid peptide (2-5 mg) in 500 μL of DI H2O. The concentration of the stock solution was determined by UV-Vis. Prior to CD measurements, stock solutions were heated to 80° C. for 10 minutes, then incubated at 4° C. for at least 48 h, followed by dilution to the predetermined concentration.

ii. Wavelength Scan

Peptide solutions (150 μM in PBS) were prepared as described above. Approximately 250 μL of peptide solution was added to a 1 mm quartz cuvette and the ellipticity was measured from 215 to 250 nm at 4° C. The measurement was repeated twice for each sample.

iii. Thermal Unfolding

Peptide solutions were heated from 4 to 80° C. with a heating rate of 0.5° C./min, during which ellipticity was monitored at 225 nm. The CD melting temperatures (Tm) were determined as the minimum of the derivative of the thermal unfolding curve. SpectraManager2 (version 2.04.00, Windows, Jasco Corporation) was used to smooth the unfolding curve (means-movement method, convolution=25), and to calculate the 1st derivative (subtract method, data points=21). The data presented is the average of two independent measurements.

iv. Refolding Rate Determination

Peptide solutions (150 μM for M-CHP and 75 μM for D-CHP) were prepared as described above. The peptide solution (250 μL) was added to a 1 mm quartz cuvette which was then capped and heated to 80° C. in a water bath for 10 min. The cuvette was quickly transferred to the CD chamber held at 4° C. and the ellipticity at 225 nm was monitored for 2 h. 100% folded was defined as the ellipticity of the peptide after incubation at 4° C. for 48 hr and 0% folded was set as the ellipticity 60 sec after placement of the cuvette in the 4° C. CD chamber (to account for changes in CD intensity caused by the temperature change).

TABLE 1
Sequences and MALDI-TOF MS of all CHPs.

| | | | m/z | m/z |
|---|---|---|---|---|
| Peptide | Sequence | | Calculated | Observed |
| M-CHP | Ac-(GPO)₆-GK-CONH₂ | [M + H⁺] | 1848.1 | 1847.9 |
| D-CHP | 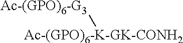 | [M + H⁺] | 3965.4 | 3963.0 |
| Biotin-M-CHP | NH₂-(GPO)₆-GGGK(Biotin)-CONH₂ | [M + H⁺] | 2146.1 | 2147.0 |
| Biotin-D-CHP | 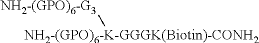 | [M + H⁺] | 4220.4 | 4216.3 |
| Biotinylated D- CHP | 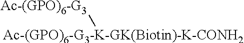 | [M + H⁺] | 4319.9 | 4318.0 |
| CF-M-CHP | CF-G₃-(GPO)₆-CONH₂ | [M + Na⁺] | 2172.0 | 2171.9 |
| CF-D-CHP | 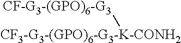 | [M + H⁺] | 4735.4 | 4734.1 |
| CF-Scrambled D-CHP | 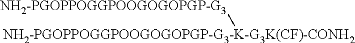 | [M + Na⁺] | 4204.4 | 4207.3 |
| Biotin- | Biotin-Ahx- | [M + H⁺] | 2792.1 | 2790.3 |
| (GPD . . . GAR) | GPDGKTGPOGPAGQDGRPGPAGPOGAR-CONH₂ | | | |
| Biotin- | Biotin-Ahx-GLTGPIGPOGPAGAOGDK-CONH₂ | [M + H⁺] | 1929.3 | 1929.0 |
| (GLT . . . GDK) | | | | |
| Biotin- | Biotin-Ahx- | [M + H⁺] | 2759.1 | 2758.3 |
| (GSO . . . GAK) | GSOGPAGPKGSOGEAGROGEAGLOGAK-CONH₂ | | | |
| Biotin- | Biotin-Ahx-GEOGPAGVQGPOGPAGEEGK-CONH₂ | [M + H⁺] | 2158.4 | 2158.0 |
| (GEO . . . GEEGK) | | | | |

5. M-CHP and D-CHP Binding to Gelatin

To prepare the crosslinked gelatin substrate, a 10% solution of porcine gelatin in PBS was heated to 80° C. for 10 min. The melted gelatin solution was pipetted into a well of a 96 well plate until the bottom of the well was completely covered, then excess solution was removed. Approximately 7 μL of the gelatin solution remained in each well. After gelatin coating, the plate was incubated at 4° C. for 15 min to allow gelatin to fully solidify. EDC-NHS crosslinking solution was produced by dissolving 192 mg EDC and 19 mg NHS in 100 mL MES buffer, and 100 μL of the crosslinking solution was added to each well and gently mixed overnight. Crosslinked films were washed at least 5 times with PBS to fully remove any remaining crosslinking solution.

Gelatin binding was assessed by adding solutions of preheated CF-M-CHP, CF-D-CHP, or CF-Scrambled D-CHP (10 μM in PBS, heated to 80° C. for 10 min) to the surface of a crosslinked gelatin film as prepared above. Wells were incubated overnight at 4° C. The wells were washed with 4° C. PBS (4×) and the fluorescence of each well was measured using a SpectraMax M2e plate reader (excitation: 492 nm, emission: 533 nm). Wells were subsequently incubated at 25° C. for 2 h, washed, and the fluorescence remeasured.

6. ELISA-Like Assay for Synthetic Collagen Fragments Binding to CHP Bound Surfaces

i. Surface Immobilization of M- and D-CHPs

A PBS solution with 10 μM of M-CHP with 100 μM glycine in PBS was prepared and heated to 80° C. for 10 min. This solution (50 μL) was added to wells in the 96 well plate which has covalent amine-capturing surface (Nunc immobilizer amino F96, VWR). Half of the 96 wells were treated with M-CHP via this method and the other half with D-CHP (0.5 μM, with 100 μM glycine in PBS) in a similar fashion. The plate was agitated at 4° C. for 2 h, solutions removed, and washed with PBS (3×). The plate was blocked with 0.1% BSA (4° C., overnight, 2×), and washed with H₂O (90° C., 10×).

ii. ELISA-Like Binding Assay

Each of the four biotin-labeled synthetic collagen peptides mimicking Rat_COL1A1 was dissolved in PBS to 25 μM. Peptide solutions were serially diluted with PBS using a 1:3 dilution factor to make 11 total solutions. These diluted solutions (50 μL) were added in triplicate to the wells of the M- and D-CHP immobilized plate. The plate was incubated at 4° C. for 2 h. Solutions were removed and the wells washed with PBS (4×). Neutravidin-HRP (50 μL, 0.4 μg/mL) was added to each well and incubated at 4° C. for 30 min. Neutravidin-HRP solution was removed and wells were washed with cold PBS (4×). Wells were developed using the Quantablu Fluorogenic Peroxidase Substrate kit (ThermoFisher). The fluorescence of each well was measured by SpectraMax M2e plate reader (excitation: 325 nm, emission: 420 nm).

iii. Curve Fitting

Intensity data was plotted on a logarithmic scale and fitted to a 4-parameter Hill slope (f=y0+a*x\hat{ }b/(c\hat{ }b+x\hat{ }b), sigmoidal, Hill, 4 parameter) using SigmaPlot 10 (Version 10.0.0.54 for Windows, Systat Software, Inc.). K_{D} was determined from the c factor of the curve fit.

7. Enrichment of Collagen Fragments in Urine from OVX and Sham-Operated Mice

i. Bone-Loss Induced by Ovariectomy

At 6-7 weeks of age, female wild type FVB mice underwent ovariectomy or sham surgery as described previously¹. Twenty-eight days after the surgery, mice were euthanized. Urine samples were collected at the time of euthanasia and the right tibia was collected for ex vivo BMD determination. Urine samples were stored at −80° C. For BMD measurements, the tibia were fixed in 10% neutral buffered formalin overnight, washed in PBS, and stored in 70% ethanol. Bone mineral density was determined using an UltraFocus DXA (Faxitron). A region including the primary and secondary spongiosa in the tibia was used to determine the BMD of the mice.

ii. CHP-Functionalized Bead Preparation

Softlink™ Soft-Release Avidin resin (150 μL of resin slurry) was added to a disposable chromatography column. The storage solution was removed and the beads were washed with PBS (4×). The solution was removed almost to dryness, and 150 μL of PBS was added to the column. A stock solution of Biotinylated D-CHP (1.19 mM) was heated to 80° C. for 10 min and 8 μL (9.5 nmol of peptide) of solution was added to the resin and mixed at 4° C. for 20 min. The resin was washed with 80° C. H₂O (10×) to dissociate and remove any CHPs that might have bound to the column. The resin was stored following manufacturer recommendation (4° C., 20% ethanol).

iii. Enrichment Procedure and Mass Search

PBS (150 μL) and urine from OVX or sham-operated mice (17.5 μL) were added to a D-CHP-functionalized column as prepared above. The column was mixed overnight at 4° C. The column was washed extensively using the following steps to remove non-specifically bound materials. The column was first rinsed with 1 mL PBS (4×). Next, the column was washed with 1 mL of a 0.1 M NaCl in 0.05% SDS solution (2×) followed by 1 mL of PBS (2×), and this cycle of washes was repeated 4 times. The column was then washed with 1 mL H₂O (4×) to remove excess detergent and salts. Collagen fragments which were bound to the column by triple helical folding were released by adding 750 μL H₂O to the column and incubating in an 80° C. water bath for 10 minutes with occasional agitation, followed by gravity elution. The elution process was repeated a second time. The elutions were combined, lyophilized, and stored at −80° C. For LC-MS/MS analysis, collections were resuspended in 50 μL of H₂O. Unenriched urine samples were prepared as follows. Urine samples (10 μL) were diluted 5-fold with H₂O and were extracted for peptide content using a ZipTip C18 column. C18 extracted solution was concentrated to approximately 50 uL by evaporation. For each LC-MS/MS run, 5 μL of concentrated solution was injected. Resulting data were assessed by a Mascot search using the parameters detailed in Table 2.

TABLE 2
Parameters for Mascot search of MS/MS data.

| Search type | MIS |
|---|---|
| Mascot version | 2.6.1 |
| Database | SwissProt |
| Fasta file | SwissProt_2017_11.fasta |
| Taxonomy filter | Rodentia (Rodents) |
| Enzyme | None |
| Maximum Missed | 2 |
| Cleavages | |
| Fixed modifications | Carbamidomethyl (C) |
| Variable modifications | Oxidation (P), Oxidation |
| | (M), Oxidation (K) |
| Peptide Mass Tolerance | 11 |
| Peptide Mass Tolerance | ppm |
| Units | |
| Fragment Mass Tolerance | 11 |
| Fragment Mass Tolerance | ppm |
| Units | |
| Mass values | Monoisotopic |
| Instrument type | ESI-QUAD-TOF |
| Isotope error mode | 1 |

iv. Overview of Sequence Matching and Clustering

All mass queries from Mascot searches which were assigned at least one amino acid sequence were assessed for similarity to collagen. To determine the fragments' collagen type of origin and map their location along the collagen sequence, each m/z assigned an amino acid sequence by the Mascot search was compared to each amino acid position along the 38 mouse collagen a chains (Table 3). A sequence was considered collagenous if the assigned peptide sequence matched the sequence from a natural collagen with fewer than one out of ten amino acids mismatching. For each match, collagen of origin, sequence position, and intensity were recorded. Some mass queries were assigned to multiple peptide sequences. In these cases, only the collagenous sequence with the highest score assigned by the Mascot search was considered. Overall collagen content was assessed by comparing the total primary ion intensity of collagenous peptides to that of all masses assigned peptide sequences. Clustering analysis was performed using the clustergram function from the bioinformatics toolbox in Matlab R2019a (Mathworks) using code developed in house which is available upon request.

TABLE 3
Collagen protein IDs used for sequence analysis.
All proteins are from Mus musculus
(Mouse) and retrieved from UniProt.

Col1a1
Col1a2
Col2a1
Col3a1
Col4a1
Col4a2
Col4a3
Col4a4
Col5a1
Col5a2
Col6a1
Col6a2
Col6a4
Col6a5
Col6a6
Col7a1
Col8a1
Col8a2
Col9a1
Col9a2
Col10a1
Col11a1
Col11a2
Col12a1
Col13a1
Col14a1
Col15a1
Col16a1
Col17a1
Col18a1
Col19a1
Col20a1
Col23a1
Col24a1
Col25a1
Col26a1
Col27a1
Col28a1

Those skilled in the art will recognize, or be able to ascertain using no more than routine experimentation, many equivalents to the specific embodiments of the method and compositions described herein. Such equivalents are intended to be encompassed by the following claims.

1. A method of enriching collagen fragments in a sample comprising
a) combining a sample comprising collagen fragments with a composition comprising a dimeric collagen hybridizing peptide (CHP),
wherein the dimeric CHP comprises a first CHP and a second CHP, one or more linkers, and a branch point,
wherein the first CHP and second CHP comprise the sequence of at least (GXY)n, wherein G is glycine, wherein X and Y are any amino acid, and wherein n is any number between 3 and 12, and
wherein the first CHP and second CHP bind to and form a triple helix with a collagen fragment; and
b) removing the bound collagen fragments from the dimeric CHP providing a product enriched with collagen fragments.

2. The method of claim 1, wherein the dimeric CHP is conjugated to a support.

3. The method of claim 2, wherein the support is beads or a multiwell plate.

4. (canceled)

5. The method of claim 1, wherein the first CHP and second CHP are identical.

6. The method of claim 1, wherein the first CHP and second CHP are different.

7. The method of claim 1, wherein X is proline, modified proline, glutamic acid, or aspartic acid.

8. The method of claim 1, wherein Y is a modified proline, lysine, or arginine.

9. The method of claim 1, wherein a glycine is modified as an Aza-glycine.

10. The method of claim 1, wherein the linker is between the collagen hybridizing peptides and the branch point.

11. The method of claim 1, wherein there are at least two linkers.

12. The method of claim 1, wherein the linker and branch point are on the C-terminal or N-terminal end of the first CHP and second CHP.

13. (canceled)

14. The method of any one of claims 1-13, wherein the linker is one or more glycine residues, aminohexanoic acid, or polyethylene glycol (PEG).

15. The method of claim 1, wherein the branch point attaches to a linker which is attached to the first CHP and to a linker which is attached to second CHP.

16. The method of any one of claims 1-15, wherein the branch point is a lysine residue.

17. The method of claim 1, wherein the dimeric CHP comprises the formula

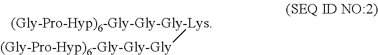

18. The peptide conjugate of claim 1, wherein the dimeric peptide comprises the formula

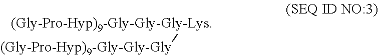

19. The method of claim 1, further comprising performing a peptidomic analysis on the product enriched with collagen fragments.

20. (canceled)

21. The method of claim 1, wherein the dimeric CHP is cyclic.

22. A method of detecting collagen in a sample comprising
a) enriching collagen fragments from a sample, wherein enriching the collagen fragments comprises
combining a sample comprising collagen fragments with a composition comprising a dimeric collagen hybridizing peptide (CHP),
wherein the dimeric CHP comprises a first CHP and a second CHP, one or more linkers, and a branch point,
wherein the collagen fragments bind the dimeric CHP; and
b) detecting the binding of the collagen fragments to the dimeric CHP.

23. (canceled)

24. A method of diagnosing a disease or injury involving collagen damage in a subject comprising
a) detecting whether collagen is present in a sample obtained from the subject,
wherein the detecting step comprises enriching collagen fragments from the sample, wherein the enriching step comprises combining the sample with a composition comprising a dimeric collagen hybridizing peptide (CHP),
wherein the dimeric CHP comprises a first CHP and a second CHP, one or more linkers, and a branch point,
wherein the first CHP and second CHP comprise the sequence of at least (GXY)n, wherein G is glycine, wherein X and Y are any amino acid, and wherein n is any number between 3 and 12, and
wherein the first CHP and second CHP bind to and form a triple helix with a collagen fragment in the sample; and
b) detecting the binding of the collagen fragments to the dimeric CHP; and
c) diagnosing the subject as having a disease or injury involving collagen damage when collagen fragments bound to the dimeric CHP are detected.

25.-31. (canceled)


SEQUENCE LISTING (7 sequences)

SEQ ID NO: 1 - protein, 150 aa, Artificial Sequence
  synthetic peptide; collagen hybridizing peptide
  misc (2)..(3): Xaa can be any amino acid
  misc (5)..(6): Xaa can be any amino acid
  misc (7)..(150): amino acids 7-150 can be present or absent
  misc (8)..(9): Xaa can be any amino acid
  misc (11)..(12): Xaa can be any amino acid
  misc (14)..(15): Xaa can be any amino acid
  misc (17)..(18): Xaa can be any amino acid
  misc (20)..(21): Xaa can be any amino acid
  misc (23)..(24): Xaa can be any amino acid
  misc (26)..(27): Xaa can be any amino acid
  misc (29)..(30): Xaa can be any amino acid
  misc (32)..(33): Xaa can be any amino acid
  misc (35)..(36): Xaa can be any amino acid
  misc (38)..(39): Xaa can be any amino acid
  misc (41)..(42): Xaa can be any amino acid
  misc (44)..(45): Xaa can be any amino acid
  misc (47)..(48): Xaa can be any amino acid
  misc (50)..(51): Xaa can be any amino acid
  misc (53)..(54): Xaa can be any amino acid
  misc (56)..(57): Xaa can be any amino acid
  misc (59)..(60): Xaa can be any amino acid
  misc (62)..(63): Xaa can be any amino acid
  misc (65)..(66): Xaa can be any amino acid
  misc (68)..(69): Xaa can be any amino acid
  misc (71)..(72): Xaa can be any amino acid
  misc (74)..(75): Xaa can be any amino acid
  misc (77)..(78): Xaa can be any amino acid
  misc (80)..(81): Xaa can be any amino acid
  misc (83)..(84): Xaa can be any amino acid
  misc (86)..(87): Xaa can be any amino acid
  misc (89)..(90): Xaa can be any amino acid
  misc (92)..(93): Xaa can be any amino acid
  misc (95)..(96): Xaa can be any amino acid
  misc (98)..(99): Xaa can be any amino acid
  misc (101)..(102): Xaa can be any amino acid
  misc (104)..(105): Xaa can be any amino acid
  misc (107)..(108): Xaa can be any amino acid
  misc (110)..(111): Xaa can be any amino acid
  misc (113)..(114): Xaa can be any amino acid
  misc (116)..(117): Xaa can be any amino acid
  misc (119)..(120): Xaa can be any amino acid
  misc (122)..(123): Xaa can be any amino acid
  misc (125)..(126): Xaa can be any amino acid
  misc (128)..(129): Xaa can be any amino acid
  misc (131)..(132): Xaa can be any amino acid
  misc (134)..(135): Xaa can be any amino acid
  misc (137)..(138): Xaa can be any amino acid
  misc (140)..(141): Xaa can be any amino acid
  misc (143)..(144): Xaa can be any amino acid
  misc (146)..(147): Xaa can be any amino acid
  misc (149)..(150): Xaa can be any amino acid
GXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXX

SEQ ID NO: 2 - protein, 43 aa, Artificial Sequence
  synthetic construct; collagen hybridizing peptide
  misc (3)..(3): Xaa is hydroxyproline
  misc (6)..(6): Xaa is hydroxyproline
  misc (9)..(9): Xaa is hydroxyproline
  misc (12)..(12): Xaa is hydroxyproline
  misc (15)..(15): Xaa is hydroxyproline
  misc_feature (18)..(18): Xaa can be any naturally occurring amino acid
  misc (23)..(23): Xaa is hydroxyproline
  misc (26)..(26): Xaa is hydroxyproline
  misc (29)..(29): Xaa is hydroxyproline
  misc (32)..(32): Xaa is hydroxyproline
  misc (35)..(35): Xaa is hydroxyproline
  misc (38)..(38): Xaa is hydroxyproline
  misc (41)..(41): Xaa is hydroxyproline
GPXGPXGPXGPXGPXGPXGGGKGGGXPGXPGXPGXPGXPGXPG

SEQ ID NO: 3 - protein, 61 aa, Artificial Sequence
  synthetic construct; collagen hybridizing peptide
  misc (3)..(3): Xaa is hydroxyproline
  misc (6)..(6): Xaa is hydroxyproline
  misc (9)..(9): Xaa is hydroxyproline
  misc (12)..(12): Xaa is hydroxyproline
  misc (15)..(15): Xaa is hydroxyproline
  misc (18)..(18): Xaa is hydroxyproline
  misc (21)..(21): Xaa is hydroxyproline
  misc (24)..(24): Xaa is hydroxyproline
  misc (27)..(27): Xaa is hydroxyproline
  misc (35)..(35): Xaa is hydroxyproline
  misc (38)..(38): Xaa is hydroxyproline
  misc (41)..(41): Xaa is hydroxyproline
  misc (44)..(44): Xaa is hydroxyproline
  misc (47)..(47): Xaa is hydroxyproline
  misc (50)..(50): Xaa is hydroxyproline
  misc (53)..(53): Xaa is hydroxyproline
  misc (56)..(56): Xaa is hydroxyproline
  misc (59)..(59): Xaa is hydroxyproline
GPXGPXGPXGPXGPXGPXGPXGPXGPXGGGKGGGXPGXPGXPGXPGXPGXPGXPGXPGXPG

SEQ ID NO: 4 - protein, 12 aa, Artificial Sequence
  synthetic construct; collagen hybridizing peptide
  misc (1)..(12): Xaa can be glycine, proline, a modified proline or aza-glycine, and at least one ofthe Xaa is aza-glycine
XXXXXXXXXXXX

SEQ ID NO: 5 - protein, 21 aa, Artificial Sequence
  synthetic construct; collagen hybridizing peptide
  misc (3)..(3): Xaa is hydroxyproline
  misc (6)..(6): Xaa is hydroxyproline
  misc (9)..(9): Xaa is hydroxyproline
  misc (10)..(10): Gly is azaglycine
  misc (12)..(12): Xaa is hydroxyproline
  misc (15)..(15): Xaa is hydroxyproline
  misc (18)..(18): Xaa is hydroxyproline
  misc (21)..(21): Xaa is hydroxyproline
GPXGPXGPXGPXGPXGPXGPX

SEQ ID NO: 6 - protein, 21 aa, Artificial Sequence
  synthetic construct; collagen hybridizing peptide
  misc (2)..(2): Xaa is hydroxyproline
  misc (5)..(5): Xaa is hydroxyproline
  misc (8)..(8): Xaa is hydroxyproline
  misc (11)..(11): Xaa is hydroxyproline
  misc (12)..(12): Gly is azaglycine
  misc (14)..(14): Xaa is hydroxyproline
  misc (17)..(17): Xaa is hydroxyproline
  misc (20)..(20): Xaa is hydroxyproline
PXGPXGPXGPXGPXGPXGPXG

SEQ ID NO: 7 - protein, 21 aa, Artificial Sequence
  synthetic construct; collagen hybridizing peptide
  misc (2)..(2): Xaa is hydroxyproline
  misc (5)..(5): Xaa is hydroxyproline
  misc (8)..(8): Xaa is hydroxyproline
  misc (12)..(12): Gly is azaglycine
  misc (14)..(14): Xaa is hydroxyproline
  misc (17)..(17): Xaa is hydroxyproline
  misc (20)..(20): Xaa is hydroxyproline
PXGPXGPXGPPGPXGPXGPXG